(19) United States
(12) Patent Application Publication
(10) Pub. No.: US 2025/0288447 A1
(43) Pub. Date: Sep. 18, 2025

(54) KNEE ORTHOSIS WITH VARUS-VALGUS ADJUSTMENT SYSTEM

(71) Applicant: OSSKIN ORTHO INC., Montréal (CA)

(72) Inventors: Pierre COUTU, Montreal (CA); Victor GRENIER, Saint-Lambert (CA); Pascale BRASSARD, Sainte-Julie (CA)

(21) Appl. No.: 19/082,443

(22) Filed: Mar. 18, 2025

Related U.S. Application Data
(60) Provisional application No. 63/566,458, filed on Mar. 18, 2024.

(51) Int. Cl. A61F 5/01

(52) U.S. Cl. CPC A61F 5/0125; A61F 2005/0155; A61F 2005/0158

(57) ABSTRACT

A knee orthosis includes a femoral cuff, a tibial cuff, and a hinge pivotally connecting the femoral cuff to the tibial cuff and configured to allow flexion-extension of the knee of the wearer. The knee orthosis includes a varus-valgus adjustment system having a single pivot defining a pivot axis extending in a sagittal plane, an actuating element such as an adjustment screw, and a translating element such as a block threadably engaged to the adjustment screw. The translating element engages a guiding feature defining a displacement path of the translating element. A varus-valgus angle is adjusted by effecting displacement of the translating element, by way of the actuating element, guided by the displacement path, thereby pivoting the respective cuff member relative to the one of the first and second hinge members about the single pivot.

### CROSS-REFERENCE TO RELATED APPLICATIONS

The present application claims priority on U.S. Patent Application No. 63/566,458 filed Mar. 18, 2024, the entire content of which is incorporated by reference herein.

### TECHNICAL FIELD

The present disclosure relates to knee orthoses.

### BACKGROUND

Knee orthoses are externally applied devices used to influence the structural and functional characteristics of the neuromuscular and skeletal systems. A knee orthosis is a brace that extends above and below the knee joint and is generally worn to support or align the knee during flexion thereof. In the case of conditions affecting the ligaments or cartilage of the knee, a knee orthosis can provide stabilization to the knee by replacing or assisting the function of these injured or damaged ligaments or cartilage of the knee. For instance, knee braces can be used to relieve pressure from the part of the knee joint affected by diseases such as arthritis or osteoarthritis by realigning the knee joint into valgus or varus. In this way, a knee orthosis may help reduce osteoarthritis pain. When used properly, a knee brace may also help a wearer to stay active by enhancing the position and movement of the knee or reducing pain.

Knee orthoses have been developed with adjustment systems configured to adjust and maintain a varus or valgus angle of the knee while the knee orthosis is worn by the wearer. Several varus-valgus adjustment systems exist and have different ways to set the varus-valgus angle of the knee, also known as offloading or unloading of the knee.

However, in spite of previous efforts, there remains room for improvement in the art of knee orthoses having a varus-valgus adjustment system.

### SUMMARY

There is accordingly provided a knee orthosis comprising: a femoral cuff configured for engaging an upper leg portion of a wearer; a tibial cuff configured for engaging a lower leg portion of the wearer; a hinge pivotally connecting the femoral cuff to the tibial cuff, the hinge including a tibial hinge member connected to a tibial cuff member of the tibial cuff; and a varus-valgus adjustment system including: a single pivot defining a pivot axis extending in a sagittal plane, the single pivot pivotally connecting the tibial hinge member to the tibial cuff member about the pivot axis; an adjustment screw rotatably connected to the tibial hinge member; and a block threadably engaged to the adjustment screw, the block comprising a protrusion projecting from the block and engaging a guide recess of the tibial cuff member or an intermediate linkage attached to the tibial cuff member; and wherein a varus-valgus angle of the tibial cuff relative to the femoral cuff is selected by rotating the adjustment screw, thereby causing the protrusion to slide in the guide recess and pivoting the tibial cuff member relative to the tibial hinge member about the single pivot between a first position and a second position different from the first position.

The knee orthosis as defined above and described herein also includes, in certain embodiments, one or more of the following features, in whole or in part, and in any combination.

In certain embodiments, the guide recess extends in a frontal plane perpendicular to the sagittal plane at an angle comprised between 20 and 40 degrees relative to a lateral direction in the frontal plane.

In certain embodiments, the adjustment screw threadably engages the block via a plurality of threads, each of the threads of the adjustment screw engaging the block being surrounded by complementarily shaped threads defined in the block.

In certain embodiments, the adjustment screw further comprises a knob for rotating the adjustment screw by hand.

In certain embodiments, the protrusion is removably connected to the block.

In certain embodiments, the knee orthosis is worn by the wearer, the single pivot is located at about a tibial plateau of the wearer.

In certain embodiments, the single pivot is located vertically below the hinge.

In certain embodiments, in the first position, the tibial cuff member is pivoted to the tibial hinge member by an angle of at most-45 degrees relative to the sagittal plane, and in the second position, the tibial cuff member is pivoted to the tibial hinge member by an angle at most of +45 degrees relative to the sagittal plane.

In certain embodiments, the tibial cuff member is pivoted to the tibial hinge member and repositionable relative thereto at any angular position between-25 degrees and +25 degrees relative to the sagittal plane.

There is also provided a knee orthosis comprising: a femoral cuff configured for engaging an upper leg portion of a wearer; a tibial cuff configured for engaging a lower leg portion of the wearer; a hinge pivotally connecting the femoral cuff to the tibial cuff, the hinge including a femoral hinge member connected to a femoral cuff member of the femoral cuff and a tibial hinge member connected to a tibial cuff member of the tibial cuff; and a varus-valgus adjustment system including a single pivot defining a pivot axis extending in a sagittal plane, the single pivot pivotally connecting one of the tibial hinge member to the tibial cuff member and the femoral hinge member to the femoral cuff member, about the pivot axis between a first position and a second position different from the first position; and when the knee orthosis is worn by the wearer, the single pivot is adapted to be located at about a tibial plateau or a femoral condyles of the wearer.

The knee orthosis as defined above and described herein also includes, in certain embodiments, one or more of the following features, in whole or in part, and in any combination.

In certain embodiments, the single pivot is located vertically below the hinge.

In certain embodiments, when worn by the wearer, the pivot axis is vertically offset from the tibial plateau by up to 10 mm.

In certain embodiments, in the first position, the tibial cuff member is pivoted to the tibial hinge member by an angle of at most −45 degrees relative to the sagittal plane, and in the second position, the tibial cuff member is pivoted to the tibial hinge member by an angle of at most +45 degrees relative to the sagittal plane.

In certain embodiments, the tibial cuff member is pivoted to the tibial hinge member and repositionable relative thereto at any angular position between −25 degrees and +25 degrees relative to the sagittal plane.

There is also provided a knee orthosis comprising: a femoral cuff configured for engaging an upper leg portion of a wearer; a tibial cuff configured for engaging a lower leg portion of the wearer; a hinge pivotally connecting the femoral cuff to the tibial cuff, the hinge including a femoral portion and a tibial portion, the tibial portion including a tibial hinge member connected to a tibial cuff member of the tibial cuff, the femoral portion and the tibial portion including corresponding shells engaged with one another and configured for pivot relative to one another, the hinge defining an axis intersecting a center of rotation of a knee of the wearer when the knee orthosis is worn by the wearer; and a varus-valgus adjustment system including: a single pivot defining a pivot axis extending in a sagittal plane, the single pivot pivotally connecting the tibial hinge member to the tibial cuff member about the pivot axis; an adjustment screw rotatably interconnecting the tibial hinge member to the tibial cuff member, the adjustment screw defining a longitudinal screw axis extending in a frontal plane orthogonal to the sagittal plane and generally parallel to the sagittal plane; and wherein a varus-valgus angle of the tibial cuff relative to the femoral cuff is selected by rotating the adjustment screw, thereby pivoting the tibial cuff member relative to the tibial hinge member about the single pivot between a first position and a second position different from the first position.

The knee orthosis as defined above and described herein also includes, in certain embodiments, one or more of the following features, in whole or in part, and in any combination.

In certain embodiments, the center of rotation is an instantaneous center of rotation at a given flexion configuration of the hinge, the instantaneous center of rotation moving along a trajectory during flexion of the hinge, and the axis intersecting the instantaneous center of rotation notwithstanding the flexion configuration of the hinge.

In certain embodiments, the trajectory is contained in a plane parallel to the sagittal plane.

In certain embodiments, when the knee orthosis is worn by the wearer, the single pivot is located at about a tibial plateau of the wearer.

In certain embodiments, the single pivot is located vertically below the hinge.

There is further provided a knee orthosis comprising: a femoral cuff configured for engaging an upper leg portion of a wearer; a tibial cuff configured for engaging a lower leg portion of the wearer; a hinge pivotally connecting the femoral cuff to the tibial cuff, the hinge including a femoral portion and a tibial portion, the femoral portion including a femoral hinge member connected to a femoral cuff member of the femoral cuff, the tibial portion including a tibial hinge member connected to a tibial cuff member of the tibial cuff; and a varus-valgus adjustment system including: a single pivot defining a pivot axis extending in a sagittal plane, the single pivot pivotally connecting either the tibial hinge member to the tibial cuff member or the femoral hinge member to the femoral cuff member for relative pivoting therebetween about the pivot axis; an adjustment screw rotatably interconnecting either the tibial hinge member to the tibial cuff member or the femoral hinge member to the femoral cuff member, the adjustment screw defining a longitudinal screw axis extending in a frontal plane orthogonal to the sagittal plane and generally parallel to the sagittal plane; and wherein a varus-valgus angle between the tibial cuff and the femoral cuff is selected by rotating the adjustment screw, thereby pivoting either the tibial cuff member relative to the tibial hinge member or the femoral cuff member relative to the femoral hinge member about the single pivot between a first position and a second position different from the first position.

There is further provided a knee orthosis for a knee comprising: a femoral cuff configured for engaging an upper leg portion of a wearer; a tibial cuff configured for engaging a lower leg portion of the wearer; a hinge pivotally connecting the femoral cuff to the tibial cuff and configured to allow flexion-extension of the knee, the hinge including a first hinge member pivotable relative to a second hinge member, the first hinge member and the second hinge member each connected to a respective cuff member of the tibial cuff and the femoral cuff; and a varus-valgus adjustment system including: a single pivot defining a pivot axis extending in a sagittal plane, the single pivot pivotally connecting one of the first and second hinge members with the respective cuff member for relative pivoting therebetween about the pivot axis; an actuating element; a translating element engaged with the actuating element, the translation element and the actuating element each being operatively connected to a respective one of: said one of the first and second hinge members; and the respective cuff member; wherein the translating element engages a guiding feature forming part of or being attached to the other of: said one of the first and second hinge members; and the respective cuff member; the guiding feature defining a displacement path of the translating element; and wherein a varus-valgus angle of the tibial cuff relative to the femoral cuff is adjusted by effecting displacement of the translating element, by way of the actuating element, guided by the displacement path, thereby pivoting the respective cuff member relative to the one of the first and second hinge members about the single pivot between a first position and a second position different from the first position.

The knee orthosis as defined above and described herein also includes, in certain embodiments, one or more of the following features, in whole or in part, and in any combination.

In certain embodiments, the actuating element includes an adjustment screw rotatably connected to the tibial hinge member.

In certain embodiments, the translating element includes a block threadably engaged to the adjustment screw, the block comprising a protrusion projecting from the block and engaging the guiding feature of the tibial cuff member or an intermediate linkage attached to the tibial cuff member, wherein the varus-valgus angle of the tibial cuff relative to the femoral cuff is selected by rotating the adjustment screw, thereby causing the protrusion to slide in the guiding feature and pivoting the tibial cuff member relative to the tibial hinge member about the single pivot between the first position and the second position different from the first position.

In certain embodiments, the guiding feature extends in a frontal plane perpendicular to the sagittal plane at an angle comprised between 20 and 40 degrees relative to a lateral direction in the frontal plane.

In certain embodiments, the adjustment screw threadably engages the block via a plurality of threads, each of the threads of the adjustment screw engaging the block being surrounded by complementarily shaped threads defined in the block.

In certain embodiments, the adjustment screw further comprises a knob for rotating the adjustment screw by hand.

In certain embodiments, the protrusion is removably connected to the block.

In certain embodiments, when the knee orthosis is worn by the wearer, the single pivot is located at about a tibial plateau of the wearer.

In certain embodiments, the single pivot is located vertically below the hinge.

In certain embodiments, in the first position, the tibial cuff member is pivoted to the tibial hinge member by an angle of at most −45 degrees relative to the sagittal plane, and in the second position, the tibial cuff member is pivoted to the tibial hinge member by an angle at most of +45 degrees relative to the sagittal plane.

In certain embodiments, the tibial cuff member is pivoted to the tibial hinge member and repositionable relative thereto at any angular position between −25 degrees and +25 degrees relative to the sagittal plane.

In certain embodiments, a locking element is operably engaged with the translating element and/or the actuating element, the locking element being operable to selectively retain the translating element in a fixed position relative to the displacement path so as to maintain a selected varus-valgus angle.

### BRIEF DESCRIPTION OF THE DRAWINGS

Reference is now made to the accompanying drawings, in which:

FIG. 1 is a perspective view of a knee orthosis in accordance with one implementation of the present technology, the knee orthosis being worn by a wearer;

FIG. 2A is a front view of the knee orthosis of FIG. 1;

FIG. 2B is a closed-up view of the knee orthosis of FIG. 2A;

FIG. 3 is a side elevation view of the knee orthosis of FIG. 1;

FIG. 4 is a closed-up, perspective view of a varus-valgus adjustment system of the knee orthosis of FIG. 1;

FIG. 5 is an exploded, perspective view of the varus-valgus adjustment system of FIG. 4;

FIG. 6A is a schematic, side elevation view of a tibial cuff member of the knee orthosis of FIG. 1;

FIGS. 6B-6D are schematic, side elevation views of a tibial cuff member in accordance with an alternate embodiment;

FIGS. 6E-6G are schematic, side elevation views of a tibial cuff member in accordance with another alternate embodiment;

FIGS. 6H-6J are schematic, side elevation views of a tibial cuff member in accordance with another alternate embodiment;

FIG. 7 is a perspective, cross-section view of the varus-valgus adjustment system of FIG. 4, with the varus-valgus adjustment system at a neutral position;

FIG. 8 is a side elevation view of the varus-valgus adjustment system of FIG. 4;

FIG. 8A is a front view of the knee orthosis of FIG. 1, with the varus-valgus adjustment system set to a valgus angle of 15 degrees;

FIG. 8B is a perspective, cross-section view of the varus-valgus adjustment system of FIG. 8A;

FIG. 9A is a front view of the knee orthosis of FIG. 1, with the varus-valgus adjustment system set to a varus angle of 15 degrees;

FIG. 9B is a perspective, cross-section view of the varus-valgus adjustment system of FIG. 9A;

FIG. 10 is a front view of the knee orthosis of FIG. 1, showing an adjustable femoral cuff;

FIG. 11 shows the trajectory in the sagittal plane along which the center of rotation (COR) travels during flexion of the knee;

FIG. 12A is a front view of a knee orthosis in accordance with an alternate embodiment, having both medial and lateral hinges;

FIG. 12B is a front view of the knee orthosis of FIG. 12A, shown with the varus-valgus adjustment system set to a valgus angle of 15 degrees;

FIG. 12C is a front view of the knee orthosis of FIG. 12A, shown with the varus-valgus adjustment system set to a varus angle of 15 degrees; and

FIG. 13 is a side view of a knee orthosis in accordance with another implementation of the present technology, the knee orthosis being worn by a wearer.

### DETAILED DESCRIPTION

The following disclosure generally describes a knee orthosis 20 being an implementation of the present technology. It is to be expressly understood that the knee orthosis 20 is merely an implementation of the present technology. The description thereof that follows is intended to be only a description of physical examples of the present technology. This description is not intended to define the scope or set forth the bounds of the present technology. In some cases, what are believed to be helpful examples of modifications to the knee orthosis 20 are also set forth hereinbelow. This is done merely as an aid to understanding, and not to define the scope or set forth the bounds of the technology. These modifications are not exhaustive, and, as a person skilled in the art would understand, other modifications are likely possible. Further, it should not be interpreted that where this has not been done, i.e., where no examples of modifications have been set forth, that no modifications are possible and/or that what is described is the sole physical means of embodying that element of the present technology. As a person skilled in the art would understand, this is likely not the case.

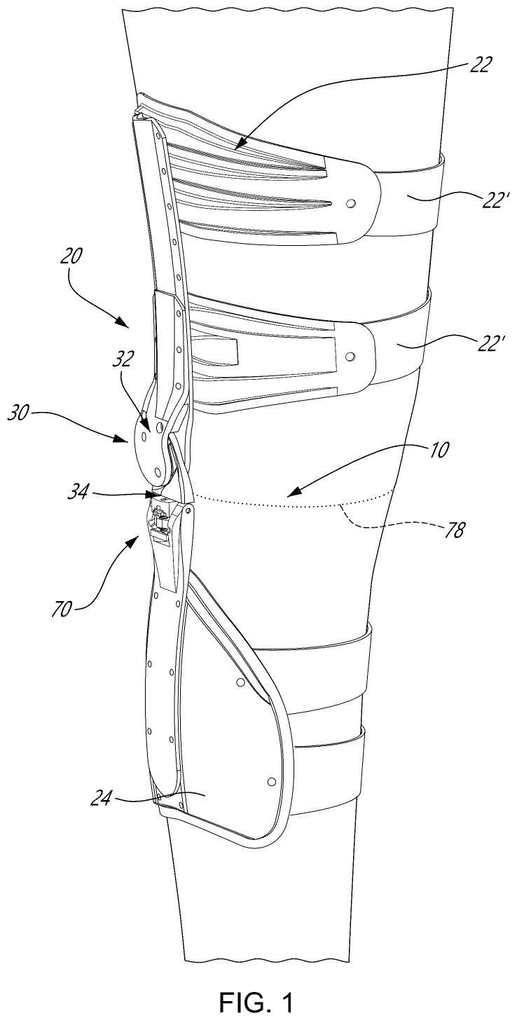

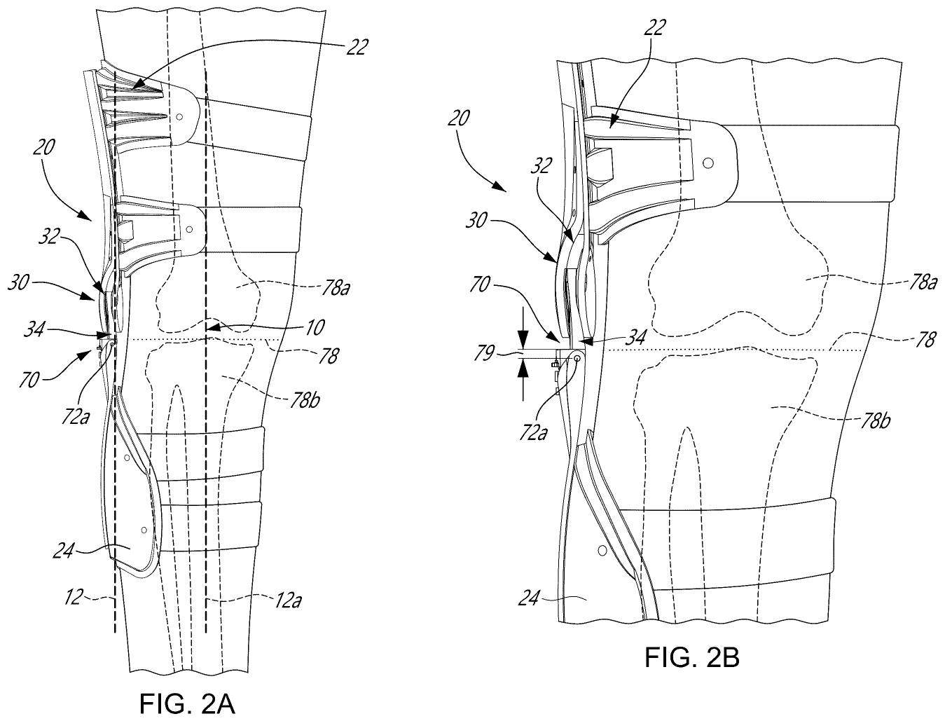

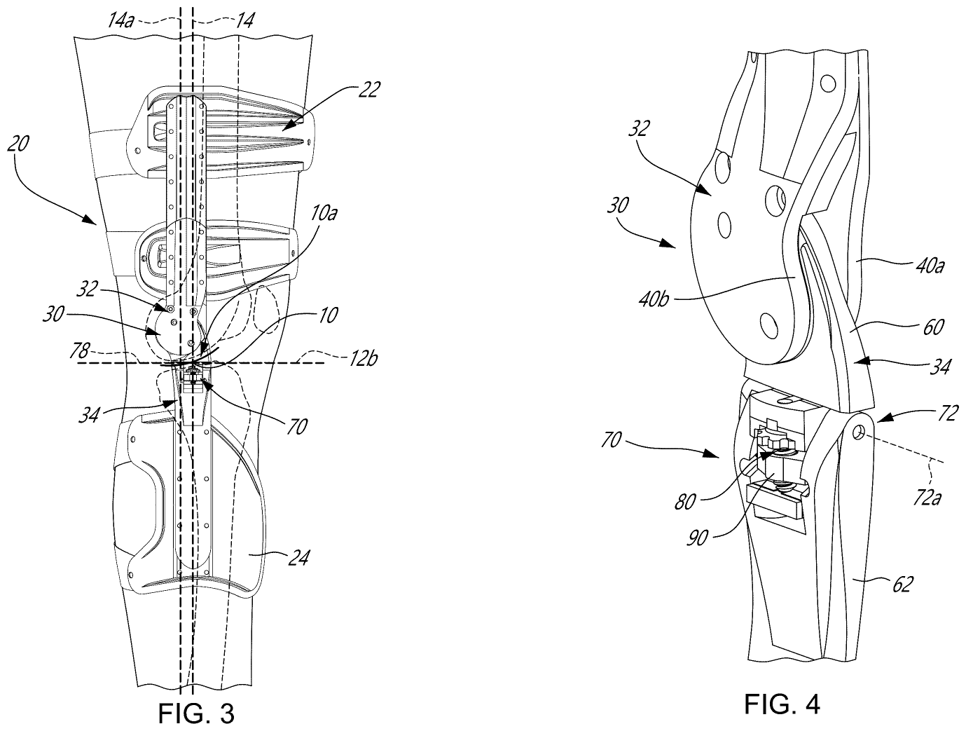

Referring to FIGS. 1 to 3, the knee orthosis 20 will be generally described. As will become apparent from the following description, the knee orthosis 20 has features designed to constrain the knee during flexion therefor and accommodates for rotation and translation kinematics in accordance with Walker's equations with respect to the knee's instantaneous flexion angle, and thus prevent movement of the knee outside a pre-determined range of motion. In other words, the knee orthosis 20 has features designed to have the knee orthosis 20 closely follow the knee's center of rotation (COR) 10. In the following description, reference is made to sagittal planes 12, 12a (FIG. 2A) and a frontal plane 14 (FIG. 3) being orthogonal to the sagittal planes 12, 12a. Sagittal plane 12 contains a pivot axis 72a (FIG. 2B) of a single pivot 72 of a varus-valgus adjustment system 70 described below, while sagittal plane 12a is parallel to the sagittal plane 12 and extends in a central region of a knee of the wearer.

The knee orthosis 20 has a femoral cuff 22 configured for engaging an upper leg portion of a wearer of the knee orthosis 20, a tibial cuff 24 configured for engaging a lower leg portion of the wearer, and a hinge 30 pivotally connecting the femoral cuff 22 to the tibial cuff 24. The hinge 30 will be described in detail below. The cuffs 22, 24 may be dimensioned and/or adapted to conform to morphologic feature(s) of the wearer. The cuffs 22, 24 may be manufactured using injection molding techniques, and be manufactured in different sizes and configurations. In some implementations, the cuffs 22, 24 are made using additive manufacturing techniques and based on a model of the wearer's knee obtained based on a scan and/or measurements of the wearer's knee. Such an implementation may provide for a custom fit of the femoral and tibial cuffs 22, 24, and may improve wearer's comfort.

In the embodiment of FIGS. 1 to 3, the knee orthosis 20 is a single hinge design, wherein the hinge 30 is located only on a lateral side of the knee orthosis 20. In other embodiments, the hinge 30 is located only on a medial side of the knee orthosis 20. In yet other embodiments, hinges 30 are located on both the medial side and the lateral side of the knee orthosis 20. The hinge 30 has a femoral portion 32 (also referred to herein as a femoral hinge member) connected to the femoral cuff 22, and a tibial portion 34 (also referred to herein as a tibial hinge member) connection to the tibial cuff 24. The femoral and tibial portions 32, 34 are connected to the corresponding cuff 22, 24 using fasteners and/or adhesives. In some implementations, the femoral and tibial portions 32, 34 are integrally formed with the corresponding femoral or tibial cuff 22, 24, and thus are integrally connected in this manner. Having the hinge 30 provided as separate components from the cuffs 22, 24 however allows for matching adequately sized or custom made cuffs 22, 24 with the hinge 30 that may be manufactured using other manufacturing techniques, such as machining or additive manufacturing, or using material(s) that differ from the one(s) forming the cuffs 22, 24. The hinge 30 allows the femoral cuff 22 and the tibial cuff 24 to pivot relative to one another during flexion of the knee. The hinge 30 is configured for allowing femoral roll back and screw home motions during flexion and extension of the knee. More particularly, referring both to FIG. 3 and FIG. 11, the femoral cuff 22 and the tibial cuff 24 pivot relative to each other about an instantaneous COR 10 that moves during flexion of the knee along a COR trajectory 10a that is contained in the sagittal plane 12a (FIG. 2A). As shown more clearly in FIG. 11, the COR 10 translates on the COR trajectory 10a along an anteroposterior axis 12b and a longitudinal axis 14a.

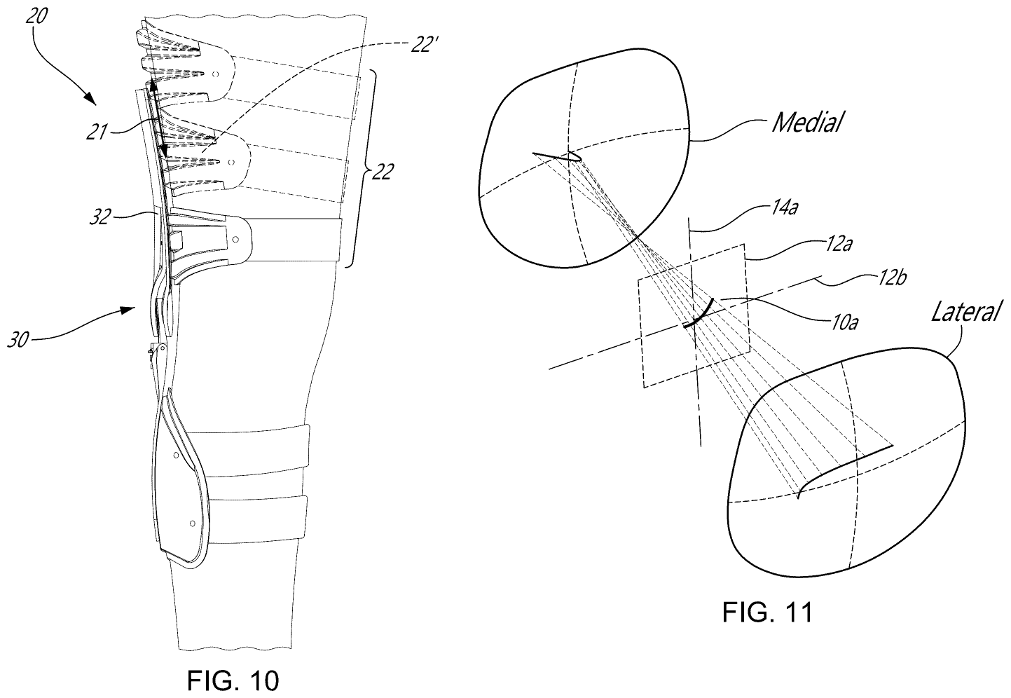

As seen in FIGS. 1-3, the femoral cuff 22 is attached to the femoral portion 32 of the hinge 30 and includes at least one (two are depicted) strap assemblies 22′ that are used to fasten the femoral cuff 22 to the upper leg of the wearer. In certain embodiments, the position(s) of these strap assemblies on the femoral cuff 22 may be fixed. However, as depicted in FIG. 10, one or more strap assemblies 22′ of the femoral cuff 22 may be engaged with the femoral portion 32 of the hinge 30 in a such a manner that the position of a strap assembly 22′ and/or a portion of the femoral cuff 22 itself can be displaced, for example along the length of the upstanding femoral portion 32 and in direction 21 as shown in FIG. 10. This selective displacement of the one or more strap assemblies 22′ and/or of a portion of the femoral cuff 22 provide adjustment for the femoral cuff 22 such that the location of the strap assemblies 22′ on the wearer's leg can be varied, as desired for optimal comfort and secure fixation of the knee orthosis 20 in place on the leg of the wearer.

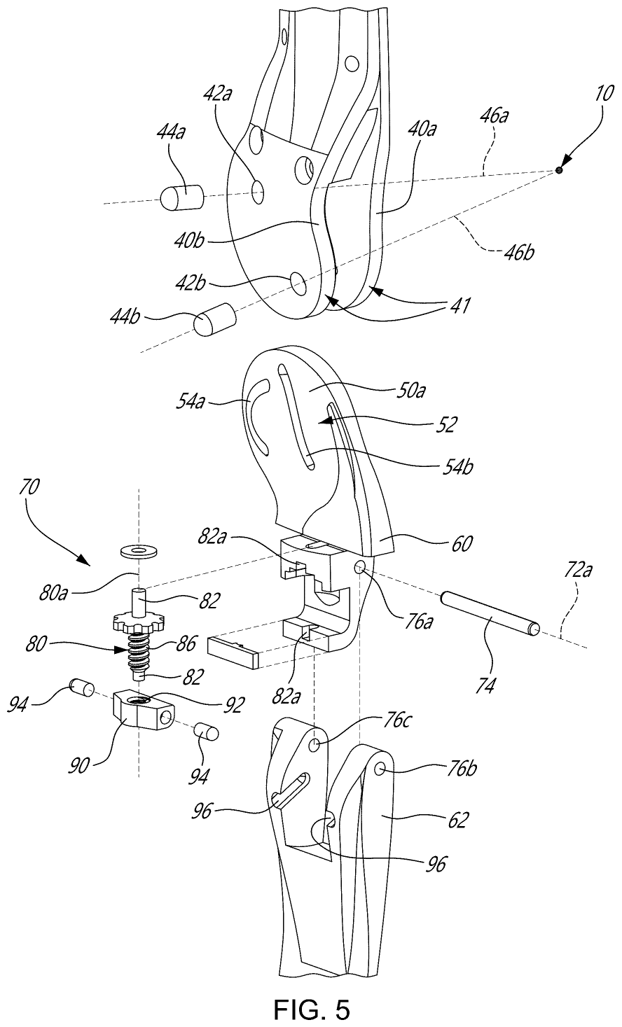

Referring to FIGS. 4 and 5, the femoral portion 32 of the hinge 30 is shown. The femoral portion 32 has spaced apart inner and outer shells 40a, 40b. While inner and outer shells 40a, 40b are shown to be integrally formed with the femoral portion 32 of the hinge, 30, it is understood that in other embodiments one or more of the inner and outer shells 40a, 40b can be formed as separate components, The inner shell 40a is located closer to the knee of the wearer, while the outer shell 40b is located further away from the knee of the wearer. The shells 40a, 40b extend parallel to one another and are offset to define a gap 40c (FIG. 5) therebetween. The shells 40a, 40b have a curved shape. Depending on the implementation, the shells 40a, 40b may have a shape corresponding to a portion of an ellipsoid or of a sphere. The shells 40a, 40b have engagement surfaces 41 adapted for engaging with other components of the hinge 30. The engagement surfaces 41 may be polished or have a surface finish adapted for reducing friction or wear when engaging other components of the hinge 30. The engagement surfaces 41 may be defined by consumable, abradable material provided on the shells 40a, 40b, in some implementations, in order to limit wear the shells 40a, 40b. The shells 40a, 40b further defines holes 42a, 42b adapted for receiving therein a corresponding pin 44a, 44b (only shown in FIG. 5). At least two pins 44a, 44b are provided. Each pin 44a, 44b extends in the gap 40c. The pins 44a, 44b are provided in the form of fasteners such as screws, rivets or bolts and nuts. Each pin 44a, 44b defines a corresponding axis 46a, 46b when inserted in the corresponding hole 42a, 42b. Other configurations are contemplated, for instance the pins 44a, 44b being integrally formed with respective shells 40a, 40b.

Still referring to FIGS. 4 and 5, the tibial portion 34 of the hinges 30 is shown. The tibial portion 34 has shell 50a adapted for insertion in the gap 40c. The shape of the shell 50a is corresponding to the shape of the shells 40a, 40b. The shell 50a further has engagement surfaces 52 adapted for sliding on the engagement surfaces 41 of the inner and outer shells 40a, 40b. The engagement surface 52 may also be polished or have a surface finish adapted for reducing friction or wear when engaging the engagement surfaces 41. The engagement surfaces 52 may also be defined by consumable, abradable material provided on the shell 50a, in some implementations, in order to limit wear of the shell 50a. In the depicted embodiment, the shell 50a further defines two guide slots 54a, 54b adapted for receiving corresponding ones of the two pins 44a, 44b therein. However, as noted in further detail below. Fewer or more guide slots and corresponding pins may alternately be used. The guide slots 54a, 54b are adapted for guiding the pivot of the femoral and tibial portions 32, 34 relative to one another during flexion of the knee orthosis 20. More particularly, the pins 44a, 44b are fixed relative to the femoral shells 40a, 40b, and the guide slots 54a, 54b allow the shell 50a to move relative to the shells 40a, 40b while being guided by the guide slots 54a, 54b. The guide slots 54a, 54b are defined by tracking projections of an instantaneous flexion axis throughout a full flexion range of the knee orthosis 20. In the present implementation, sidewalls of the tibial portion 34 defining the guide slots 54a, 54b extend parallel to the corresponding axis 46a, 46b. In other implementations, the sidewalls could have a convex, arcuate shape to allow rocking movement of the pins 44a, 44b when moving within the corresponding guide slot 54a, 54b. It is also contemplated that there could be more or less than two guide slots and corresponding pins in other implementations, depending on the type and amplitude of motion that the knee orthosis 20 is designed to accommodate or restrain. It is also contemplated that the guide slots and pins can be integrated into any mixed configurations, where the guide slots and pins are integrated in either femoral or tibial shells, or a combination of the two. More particularly, in an alternate embodiment it is contemplated that the hinge requires at least one guide slot and at least one corresponding pin or other suitable protrusion that is received within the guide slot. The guide slot(s) and pin(s) can also be integrated into any positional configurations, or permutations, meaning any guide slot can be integrated into either the tibial or femoral shell, with the corresponding pin integrated to the opposing shell.

Referring to FIG. 5, there are shown the axes 46a, 46b extending from the hinge 30 toward the COR 10 of the knee. More particularly, in FIGS. 4 and 5, the hinge 30 is at 0° of flexion, and the axes 46a, 46b converge and intersect the COR 10 of the knee for this instantaneous flexion angle. The instantaneous COR 10 moves along the trajectory 10a contained in the sagittal plane 12a during flexion of the hinge 30, and the axes 46a, 46b converge and intersect the instantaneous COR 10 notwithstanding the flexion angle of the hinge 30. Put differently, the shape of the shells 40a, 40b, 50a and the configuration of the pins 44a, 44b and guide slots 54a, 54b allow for the hinge 30 to follow the knee's COR 10 throughout a range of flexion of the knee orthosis 20. This feature may assist the knee orthosis 20 in allowing femoral roll back and screw home motions during flexion and extension of the knee.

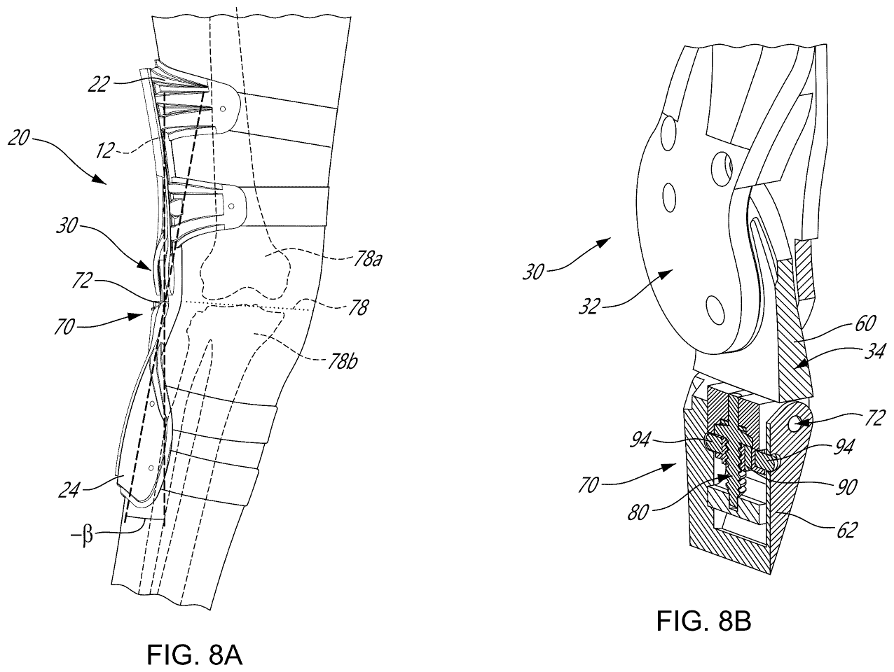

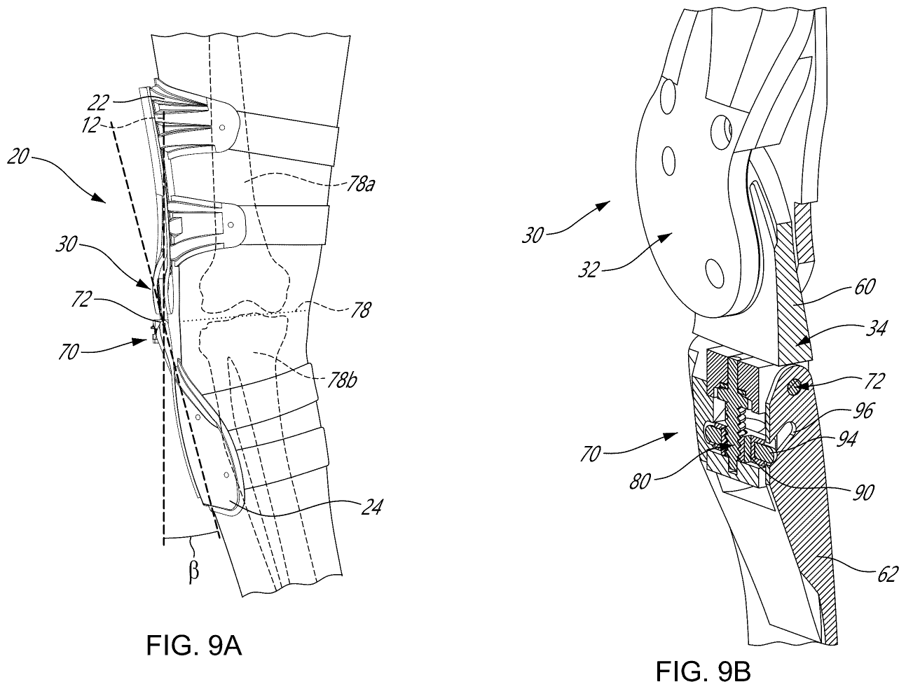

Referring now to FIGS. 4 to 8, the tibial portion 34 of the hinge 30 further includes a tibial hinge member 60 connected to a tibial cuff member 62. The tibial cuff member 62 is connected to the tibial cuff 24 using fasteners and/or adhesives, as described above. The knee orthosis 20 further includes a varus-valgus adjustment system 70 provided between the tibial portion 34 of the hinge 30 and the tibial cuff 24. As will become apparent from the following description and as best shown in FIGS. 8A to 9B, the varus-valgus adjustment system 70 is adapted for moving the tibial cuff 24 relative to the femoral cuff 22 to unload the knee of the wearer. In other embodiments, the varus-valgus adjustment system 70 is provided between the femoral portion 32 of the hinge 30 and the femoral cuff 22, with the varus-valgus adjustment system 70 adapted for moving the femoral cuff 22 relative to the tibial cuff 24 to unload the knee of the wearer.

Referring to FIGS. 2A, 2B, 4 and 5, the varus-valgus adjustment system 70 includes a single pivot 72 defining a pivot axis 72a extending in the sagittal plane 12. The single pivot 72 pivotally connects the tibial hinge member 60 to the tibial cuff member 62 about the pivot axis 72a. More particularly, the single pivot 72 is defined by a pin 74 (FIG. 5) extending in aperture 76a defined in the tibial hinge member 60 and in apertures 76b, 76c defined in the tibial cuff member 62. The pin 74 may be retained in the apertures 76a, 76b, 76c by press-fit, using circlips, or any other suitable means.

When the knee orthosis 20 is worn by the wearer, as shown in FIGS. 1 to 3, the single pivot 72 is located at about a tibial plateau 78 of the wearer, and vertically below the hinge 30. The tibial plateau 78 is defined at the junction of the femur 78a and tibia 78b of the wearer. In the context of the present description, the expression “at about a tibial plateau 78 of the wearer” means to be as close as possible, in a vertical direction, to the tibial plateau 78 of the wearer. As best seen in FIG. 2B, the pivot axis 72a is vertically offset from the tibial plateau 78 by a distance 79 of 4.5 mm, and could be vertically offset from the tibial plateau 78 by a distance 79 of up to 10 mm. Other implementations are contemplated where the pivot axis 72a could be vertically offset from the tibial plateau 78 by a distance 79 greater than 10 mm. In the embodiment where the varus-valgus adjustment system 70 is provided between the femoral portion 32 of the hinge 30 and the femoral cuff 22, the single pivot is located at about a femoral condyles of the wearer, and vertically above the hinge 30.

Referring back to FIGS. 4 and 5, the varus-valgus adjustment system 70 further includes an actuating element, illustratively an adjustment screw 80 rotatably connected to the tibial hinge member 60. The adjustment screw 80 has threadless ends 82 adapted for being received and retained within complementarily shaped recesses 82a defined in the tibial hinge member 60. In other words, when the adjustment screw 80 is retained within the recesses 82a, the adjustment screw 80 can still freely rotate. In the depicted embodiment, the adjustment screw 80 extends generally vertically in the frontal plane 14, i.e., a longitudinal screw axis 80a of the adjustment screw 80 is contained in the frontal plane 14, and extends generally parallel to the sagittal plane 12. Put differently, the longitudinal screw axis 80a does not extend generally perpendicular or transverse to the sagittal plane 12 and to the frontal plane 14. In alternate embodiments, however, the adjustment screw 80 can also extend at an angle relative to the frontal plane. The adjustment screw 80 further includes a knob 84 for rotating the adjustment screw 80 by hand. Put differently, no tool is required to rotate the adjustment screw 80. In other implementations, the adjustment screw 80 could have features for being rotated with a tool, such as a screwdriver or a hexagonal key. The adjustment screw 80 also has threads 86 defined thereon. Other actuating elements for imparting motion to the translating element can be contemplated, for instance an actuating element that incorporate a rack (engaging a corresponding pinion of the translating element), a rigid bar, a hydraulic piston, or a cam (engaging a corresponding push rod of the translating element).

Referring to FIGS. 4 to 8, the varus-valgus adjustment system 70 further includes a translating element engaged with the actuating element. In certain embodiment a locking element may also be provided, the locking element being in operative engagement with the translating element and/or the actuating element, and operable to selectively retain (or “lock”) the translating element in a given fixed position such as to maintain a desired varus-valgus angle. Such a locking element serves to lock the translating element in a desired position, such as to retain a fixed varus-valgus angle. However, in cases where either the translating element or the actuating element itself incorporates a locking feature (as is the case with the adjustment screw 80), either inherently or as an additional feature, then a separate locking element may not be required. In all cases, however, the actuating element is adapted to impart motion to the translation element to cause the tibial cuff member 62 to pivot relative to the tibial hinge member 60. Illustratively, the translating element in this embodiment includes a block 90 threadably engaged to the adjustment screw 80. The block 90 defines threads 92 (FIG. 5) shaped complementarily to the threads 86 of the adjustment screw 80. In this embodiment, therefore, the irreversible nature of the adjustment screw and translating block 90 serves itself as a locking feature, in that when the adjustment screw is no longer being rotated, the block 90 will remain fixed in its relative position thereon. As mentioned, above, in other alternative embodiments (e.g., if the varus-valgus adjustment system 70 includes another type of mechanism, such as a rack or cam mechanism for example), then an additional locking element is provided to retain the two relatively-movable components in fixed relative position when a desired varus-valgus angle has been reached. In certain embodiments, this additional locking element can include for example a ratchet, a spring plunger, a spring-loaded wedge, mated male/female teeth engagement, and the like.

When the adjustment screw 80 is threadably engaged to the block 90, each of the threads 86 of the adjustment screw 80 engaging the block 90 is surrounded by complementarily shaped threads 92. In other words, each of the threads 86 of the adjustment screw 80 engaging the block 90 engages the complementarily shaped threads 92 over 360 degrees. Put differently, the engagement of the adjustment screw 80 with the block 90 is over a plurality of threads 86, 92, which better distributes the load applied to the varus-valgus adjustment system 70. In addition, the improved distribution of the load applied to the varus-valgus adjustment system 70 over the threads 86, 92 may provide for improved durability over other varus-valgus adjustment systems where only a portion of a thread of an adjustment screw engages/meshes with another component, such as a gear, of such a varus-valgus adjustment system. As discussed in further detail below, the translating element (e.g., the block 90) engages a guiding feature (e.g., the guide recess 96) of the tibial cuff member 62, the guiding feature defining a guide path along which the translating element is adapted to translate. The guiding feature may include the guide recess 96, as in this embodiment, or an intermediate linkage as will be described in the examples provided below. Other translating elements can be contemplated, for instance a translating element including a pinion (engaging a corresponding rack of the actuating element), a sliding block, a hydraulic piston, or a push rod (engaging a corresponding cam of the actuating element).

The block 90 further includes protrusions 94 projecting from the block 90 (FIG. 5). The protrusions 94 are, in the present implementation, pins removably connected to the block 90 by press fit. Since the protrusions 94 are removable from the block 90, they can be replaced if, for example, they are worn out. It is contemplated that, in other implementations, the protrusions 94 could be connected to the block 90 in an unmovable manner, for example by being integrally formed with the block 90. The protrusions 94 engage corresponding guide recesses 96 of the tibial cuff member 62.

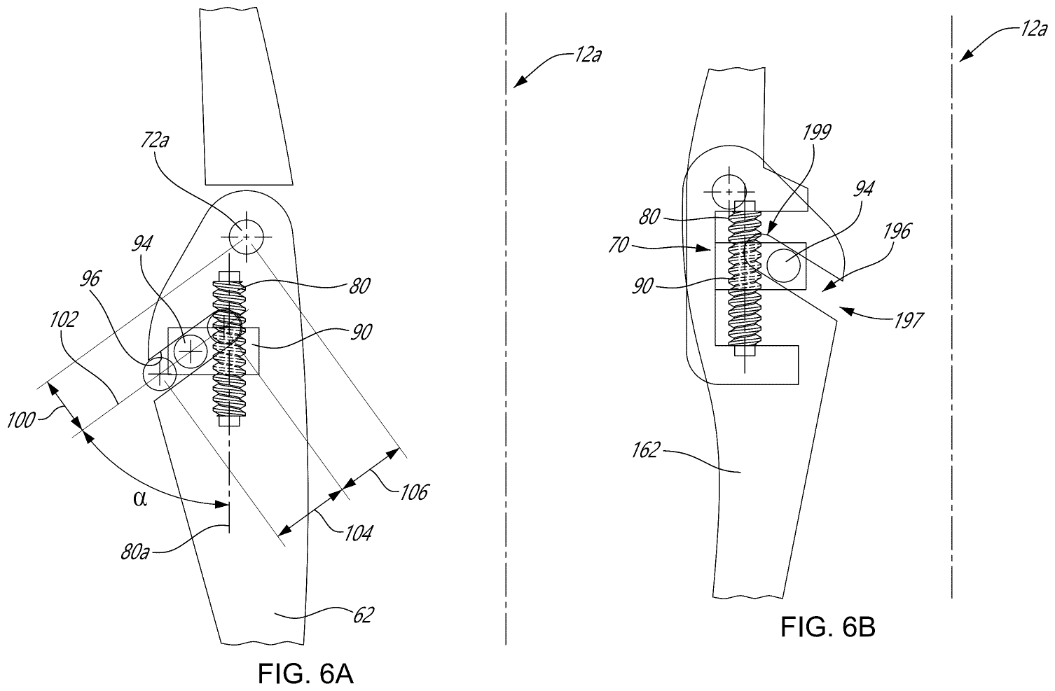

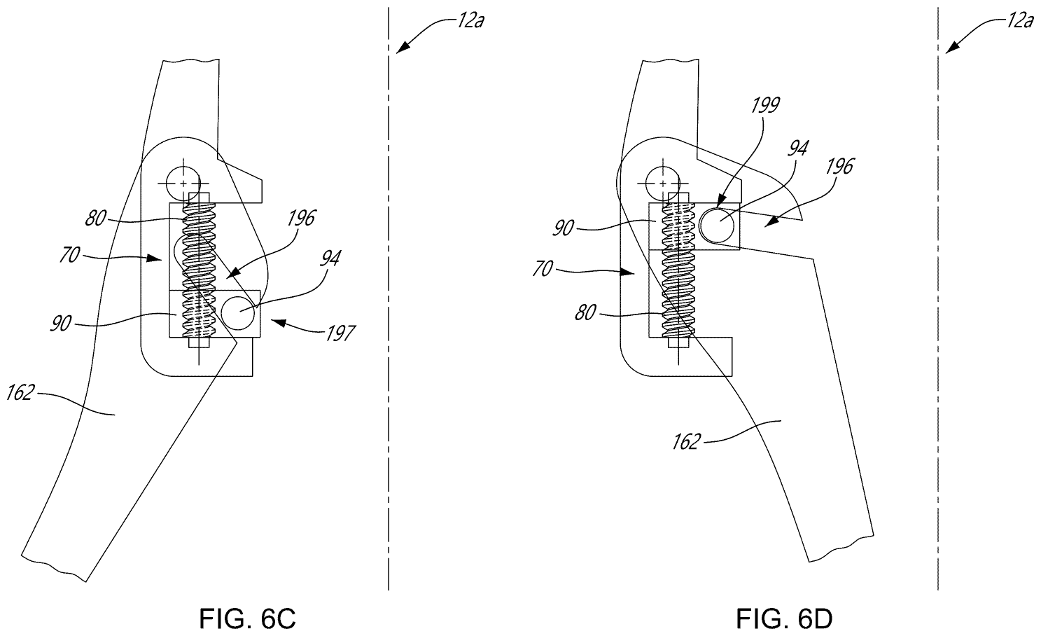

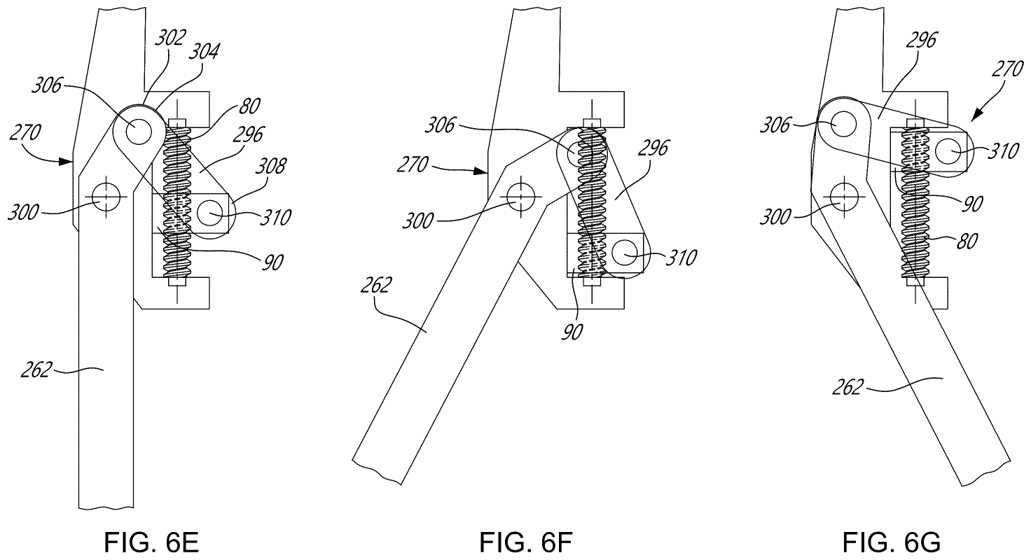

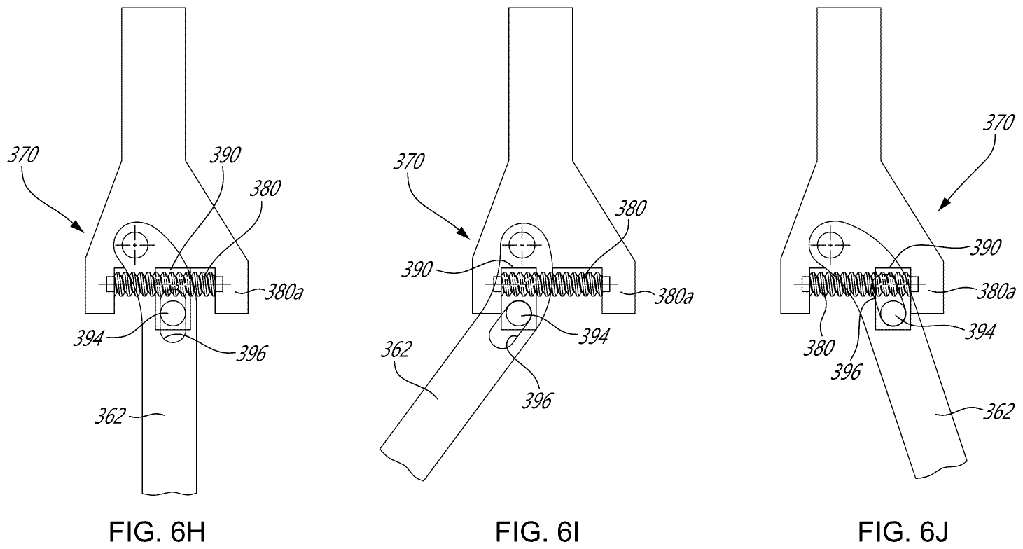

As best seen in FIG. 6A, each one of the guide recesses 96 (which act in this embodiment as the guiding feature) extends at an angle α relative to the longitudinal screw axis 80a contained in the frontal plane 14 (i.e. the frontal plane 14 is contained in the sheet of FIG. 6A). The angle α of the guide recesses 96 can be any suitable angle between 0 and 90 degrees, although in certain embodiments the angle α ranges between 25 and 75 degrees, and more preferably between 30 and 60 degrees. The angle α of the guide recesses 96 depicted in FIG. 6A is approximate 45 degrees. In all embodiments, however, the guide recess centerline 102 and the screw axis 80a cannot be parallel. In yet other implementations, the guide recesses 96 could also have a curved or non-linear profile, in which case the guide recess centerline 102 (which may for example define the guide path or displacement path relative to which the block is displaced) will similarly have the same curved or non-linear shape. More particularly, having the guide recesses 96 being straight as shown for instance in FIG. 6A allows for a varying “speed” of angular adjustment per revolution of the adjustment screw 80 throughout the range of adjustment, while a non-linear profile of the guide recesses 96 could compensate for this effect and allow for a constant correction angle per revolution of the adjustment screw 80.

FIG. 6B depicts an alternate configuration, wherein the tibial cuff member 162 is flipped around, such that the guides recesses 196 in the tibial cuff member 162 are angled at a similar angle α but faces in the opposite direction. Accordingly, the guide recesses 196 extend from an open, lowermost, end 197 of the guide recesses 196 that is located closer to the sagittal plane 12a, to a closed, uppermost, end 199 of the guide recesses 196 that is located further away from the sagittal plane 12a. This is opposite to the configuration of the guide recesses 96 of the tibial cuff member 62 as shown. The tibial cuff member 162 of FIG. 6B otherwise operates as per the tibial cuff member 62 described above, and is positioned with the same varus-valgus adjustment system 70. More particularly, the varus-valgus adjustment system 70 includes a block 90 threadably engaged to the adjustment screw 80, and the protrusions 94 which extend from the block 90 engage the corresponding guide recesses 196 of the tibial cuff member 162 for sliding engagement therein.

FIG. 6B shows the tibial cuff member 162 in a relatively neutral or mid-range position of varus-valgus. For completeness of understanding, FIG. 6C shows the same tibial cuff member 162 in a maximum valgus position, whereas FIG. 6D shows the tibial cuff member 162 in a maximum varus position. As seen in FIG. 6C, in the maximum valgus position, the protrusions 94 of the block 90 are located at the lowermost open end 197 of the guide recesses 196 in the tibial cuff member 162. As seen in FIG. 6D, in the maximum varus position, the protrusions 94 of the block 90 are located at the uppermost closed end 199 of the guide recesses 196 in the tibial cuff member 162.

It can thus be said that various embodiments of the disclosed knee orthosis 20 include a femoral cuff 22 configured for engaging an upper leg portion of a wearer, a tibial cuff 24 configured for engaging a lower leg portion of the wearer, and a hinge 30 pivotally connecting the femoral cuff 22 to the tibial cuff 24 to allow flexion-extension of the knee. The hinge 30 includes a first hinge member pivotable relative to a second hinge member. For instance, in certain embodiments, the first hinge member is a tibial hinge member 34 and the second hinge member is a femoral hinge member 32. The opposite arrangement is possible as well, i.e., the first hinge member is the femoral hinge member 32 and the second hinge member is the tibial hinge member 34. The first hinge member and the second hinge member are each connected to a respective cuff member of the tibial cuff 24 and the femoral cuff 22. The knee orthosis 20 further includes a varus-valgus adjustment system 70 with a single pivot 72 defining a pivot axis 72a extending in a sagittal plane 12 The single pivot 72 pivotally connects one of the first and second hinge members with the respective cuff member for relative pivoting therebetween about the pivot axis 72a. The varus-valgus adjustment system 70 further includes an actuating element and a translating element engaged with the actuating element. The translation element and the actuating element are each operatively connected to a respective one of: said one of the first and second hinge members; and the respective cuff member. The translating element engages a guiding feature forming part of or being attached to the other of the one of the first and second hinge members and the respective cuff member. The guiding feature defines a displacement path along which the translating element is configured to translate. A varus-valgus angle of the tibial cuff 24 relative to the femoral cuff 22 is adjusted by effecting displacement of the translating element, by way of the actuating element, guided by the displacement path, thereby pivoting the respective cuff member relative to the one of the first and second hinge members about the single pivot 72 between a first position and a second position different from the first position. Various additions and modifications to the above-described knee orthosis are contemplated.

It will be appreciated that alternate configurations of the tibial cuff member and/or the varus-valgus adjustment mechanism thereof are also possible, without departing from the general function and operation of the tibial cuff members described above, which similarly permit adjustment of the varus-valgus angle.

FIGS. 6E-6G depict such an alternate configuration. More particularly, FIGS. 6E-6G depict an alternate varus-valgus adjustment system 270, which is used to adjust the angular position of the tibial cuff member 262. FIG. 6E shows the tibial cuff member 262 in a relatively neutral or mid-range position of varus-valgus. FIG. 6F shows the tibial cuff member 262 in a maximum valgus position, whereas FIG. 6G shows the tibial cuff member 262 in a maximum varus position.

The varus-valgus adjustment mechanism 270 operates in a manner similar to the varus-valgus adjustment mechanism 70, however as will be appreciated from FIGS. 6E-6G, rather than including any guide recesses, the tibial cuff member 262 is interconnected with displaceable block 90 (i.e., the translating element in this embodiment) of the varus-valgus adjustment mechanism 270 via an intermediate linkage 296 (which act in this embodiment as the guiding feature). The tibial cuff member 262 pivots around a first pivot 300. The upper end 302 of the tibial cuff member 262 is pivotably connected to an upper end 304 of the intermediate linkage 296 by a second pivot 306. A lower end 308 of the intermediate linkage 296 is pivotably connected with the sliding block 90 via a third pivot 310. With the varus-valgus adjustment mechanism 270, rotating the screw 80 (which acts as the actuating element in this embodiment) will cause the block 90 (the translating element in this embodiment) to be displaced either upward (toward the maximum varus position as shown in FIG. 6G) or downward (toward the maximum valgus position as shown in FIG. 6F)—depending on the direction of rotation of the screw 80.

FIGS. 6H-6J depict yet another possible configuration of the varus-valgus adjustment mechanism of the present disclosure. The varus-valgus adjustment mechanism 370 of FIGS. 6H-6J is similarly used to adjust the angular position of the tibial cuff member 362. FIG. 6H shows the varus-valgus adjustment mechanism 370 and the tibial cuff member 362 in a neutral or mid-range position of varus-valgus. FIG. 6I shows varus-valgus adjustment mechanism 370 and the tibial cuff member 362 in a maximum valgus position, whereas FIG. 6J shows the varus-valgus adjustment mechanism 370 and the tibial cuff member 262 in a maximum varus position.

The varus-valgus adjustment mechanism 370 operates in a manner similar to the varus-valgus adjustment mechanism 70 described above, however as will be appreciated from FIGS. 6H-6J, the tibial cuff member 362 is interconnected with a displaceable block 390 of the varus-valgus adjustment mechanism 370 that translates laterally, for example horizontally. More particularly, the displaceable block 390 (i.e., the translating element in this embodiment) is threadably engaged with the adjustment screw 380 (i.e., the actuating element in this embodiment) that extends generally horizontally, rather than vertically. As the adjustment screw 380 is rotated, therefore, the displaceable block will be displaced along the length of the adjustment screw 380 and thus translates along the screw axis 380a of the adjustment screw 380.

Much like in the varus-valgus adjustment mechanism 70 described above, the displaceable block 390 of the varus-valgus adjustment mechanism 370 includes a guiding feature comprising a protrusion 394 (e.g., a pin) therein that is received in, and can slide within, a cooperating guide recess 396 formed in the tibial cuff member 362. In this embodiment, however, the guide recess 396 (i.e., part of the guiding feature in this embodiment) may form a slot having closed ends and which has a closed perimeter. In a particular embodiment, the guide recess 396 may extend along the tibial cuff member 362 in a direction that may substantially correspond to a longitudinal direction of the tibial cuff member 362. In the depicted embodiment, the guide recess 396 extends in a longitudinal direction that is substantially normal to the screw axis 380a, when the varus-valgus adjustment mechanism 370 and the tibial cuff member 362 are in their neutral or mid-range position of varus-valgus (FIG. 6H). Other guiding features are contemplated, for instance that includes linkage 296.

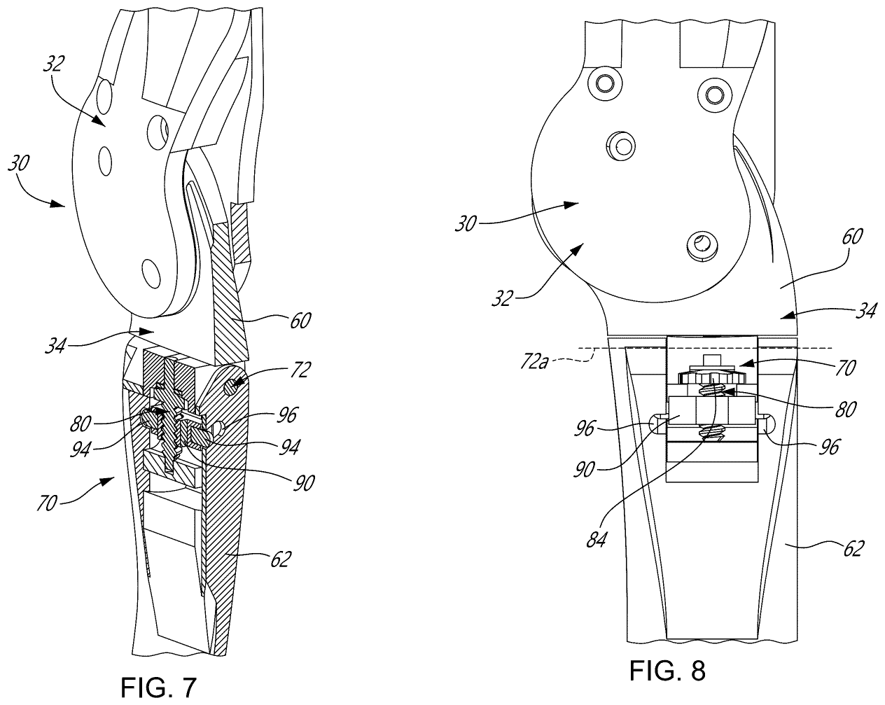

Referring now generally to FIGS. 7 to 9B, adjustment of varus-valgus angle with the varus-valgus adjustment system 70 as defined above will now be described in further detail.

The varus-valgus angle of the tibial cuff 24 relative to the femoral cuff 22 is selected or adjusted by rotating the adjustment screw 80, thereby causing the protrusions 94 to slide in their corresponding guide recess 96 and pivoting the tibial cuff member 62 relative to the tibial hinge member 60 about the single pivot 72 between a first position (for example, the position shown in FIG. 8A) and a second position (for example, the position shown in FIG. 9A) different from the first position.

The positioning and arrangement of the pivot axis 72a and of the guide recesses 96 is selected to define the varus-valgus adjustment range that is permitted by the varus-valgus adjustment system 70. For example, should more or less varus-valgus adjustment range be desired, a designer of the knee orthosis 20 can vary the angle α, a perpendicular distance 100 between the pivot axis 72a and a guide recess centerline 102, guide recess length 104 and distance 106 to obtain the desired varus-valgus adjustment range. Furthermore, should more or less varus-valgus adjustment range be desired, a designer of the knee orthosis 20 can vary the length of the adjustment screw 80 and/or the guide recess length 104. Therefore, other implementations are contemplated where such design parameters are varied from the present implementation.

The varus-valgus adjustment system 70 provides for positions ranging between −45 degrees (i.e., angle −β in FIG. 8A) relative to the sagittal plane 12 (i.e., 45 degrees in valgus), and +45 degrees (i.e., angle β in FIG. 9A) relative to the sagittal plane 12 (i.e., 45 degrees in varus). In another embodiment, the varus-valgus adjustment system 70 provides for positions between −35 degrees (i.e., 35 degrees in valgus) and +35 degrees (i.e., 35 degrees in varus). Alternately still, and in in the implementation shown, the varus-valgus the adjustment system 70 provides for positions between −25 degrees (i.e., 25 degrees in valgus) and +25 degrees (i.e., 35 degrees in varus). In FIGS. 8A-8B, an angle of −15 degrees relative to the sagittal plane 12 is (i.e., 15 degrees in valgus) is shown, and in FIGS. 9A-9B, an angle of +15 degrees (i.e., 15 degrees in varus) is shown. It is to be understood that an angle β of 0 degrees means that the varus-valgus is neutral. Accordingly, an angle β of 0 degrees is within the ranges of adjustment possible, as noted above. Other implementations having different range of positions and motion are also contemplated, for example ones where different maximum varus and maximum valgus angular positions are employed.

By having the single pivot 72 and the pivot axis 72a located at about the tibial plateau 78 of the wearer, the varus-valgus adjustment system 70 and the knee orthosis 20 permit that the forces required to be applied on the leg to unload the knee are reduced compared to other knee orthoses having different varus-valgus adjustment systems. Put differently, the position of the pivot axis 72a being at about the tibial plateau 78 allows the application of force closer to the most lax/flexible region along the leg of the wearer, i.e., the joint/inter-bony plane located at the tibial plateau 78. Advantageously, by using the knee orthosis 20 and the varus-valgus adjustment system 70, the forces required to correct for varus-valgus alignment are expected to be reduced compared to other knee orthoses having different varus-valgus adjustment systems, thus reducing risks of discomfort and pressure points for the wearer. In other words, the position of the pivot axis 72a located at about the tibial plateau allows for angular correction of the pathological leg where the misalignment initially occurs between the bones at the tibial plateau 78. The depicted knee orthosis 20 utilizes three point unloading to reduce pressure inside the wearer's knee. In particular, the femoral cuff 22 and tibial cuff 24 provide two points of leverage, in a first direction, applied respectively towards the thigh and calf of the wearer. The hinge 30 and varus-valgus adjustment system 70 provide a third point of leverage, towards the femoral condyle, acting in a direction opposite the first two points of leverage. In another embodiment, the knee orthosis 20 utilizes four point unloading to reduce pressure inside the wearer's knee, wherein the hinge 30 and varus-valgus adjustment system 70 apply two distinct points of leverage, i.e., above and below the femoral and tibial condyles, rather than a single point of leverage. This configuration (i.e., a four-point unloading configuration) can in some cases reduce discomfort that would otherwise be caused by pressure applied on the femoral condyle, which is know to be sensitive.

As noted above, the knee orthosis 20 as depicted in FIGS. 1 to 3 for example, is a single hinge design, wherein the hinge 30 is located only on a lateral side of the knee orthosis 20. However, it will also be appreciated that the varus-valgus adjustment systems as described above can also be implemented in a symmetrical or “double upright” design, wherein two hinges are provided, one on each side of the leg/knee. It will also be appreciated that the varus-valgus adjustments systems 70 described above can also include a single hinge 30 located only on a medial side of the knee orthosis 20.

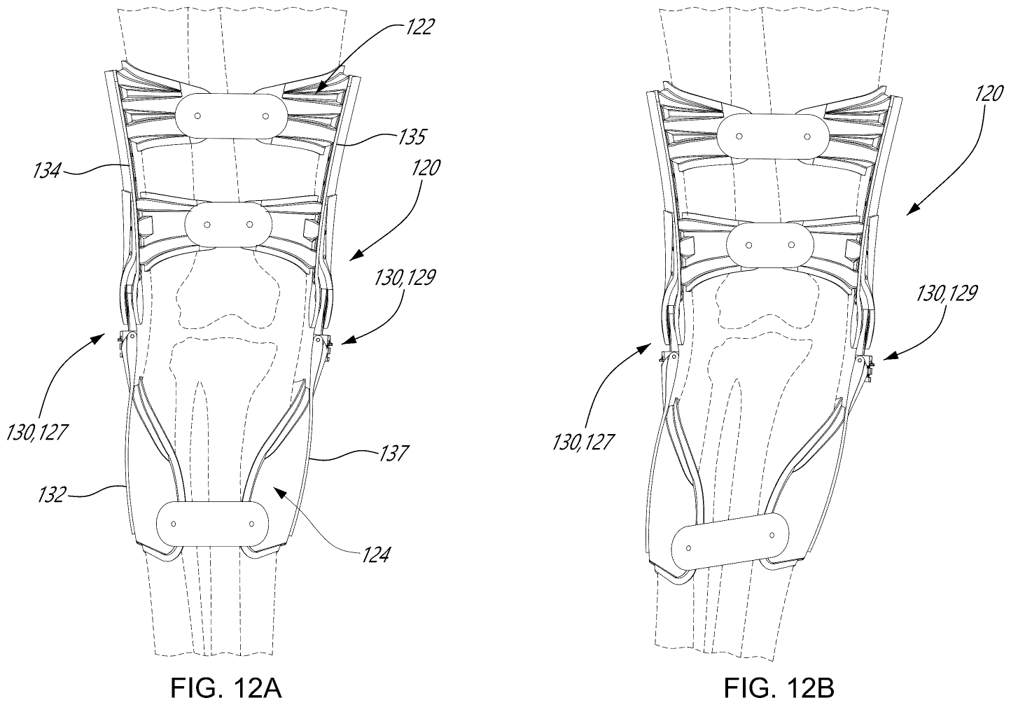

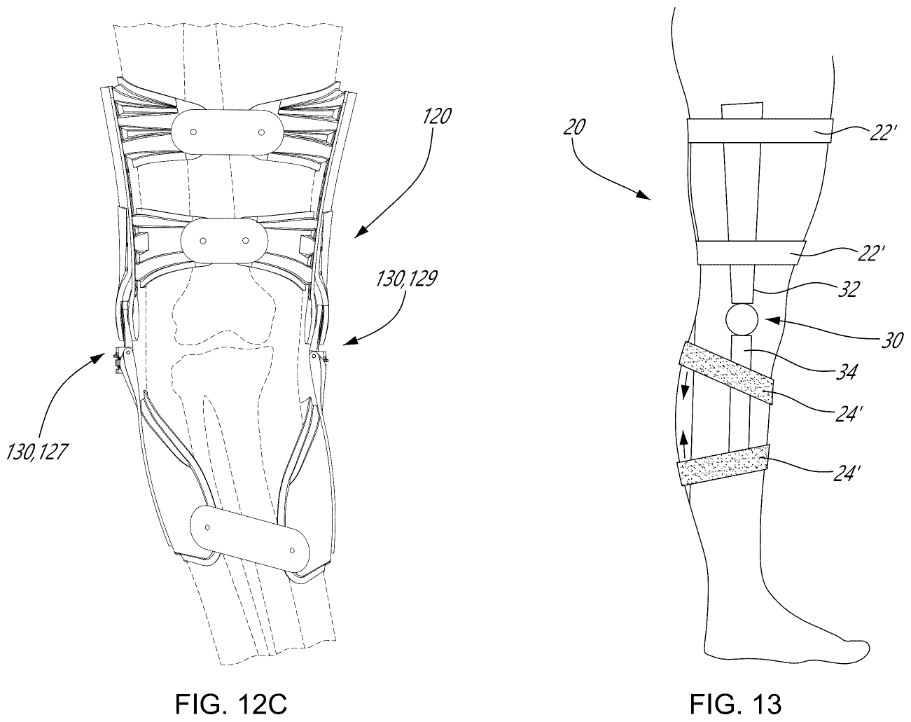

Referring now to FIG. 12A-12C, such an alternate embodiment is depicted. The knee orthosis 120 as shown in FIGS. 12A-12C has two hinges 130, namely a lateral hinge 127 configured to be mounted on the lateral side of the wearer's leg and a medial hinge 129 configured to be mounted on the medial side of the wearer's leg. The lateral hinge 127 includes an upstanding femoral portion 134 and the medial hinge 129 includes an upstanding femoral portion 135, which are interconnected by a femoral cuff 122. Similarly, the lateral hinge 127 includes a downwardly extending tibial portion 132 and the medial hinge 129 includes a downwardly extending tibial portion 137, which are interconnected by a tibial cuff 124. Despite this symmetrical or “double upright” design, the hinges 130 of the knee orthosis 120 operate as per the hinges 30 described above, and therefore include a varus-valgus adjustment system 70, 270, 370 as described herein.

The reduction of the forces required to unload the knee may improve wearer's comfort and reduce pressure points created by the knee orthosis 20 compared to other knee orthoses having different varus-valgus adjustment systems, and notably compared to the other knee orthoses having a varus-valgus adjustment system modifying the varus-valgus angle above the distal femur which generate a bending force between the distal femur and the femoral shank. Such a bending force is considered less effective as it is applied on a single rigid body (i.e., the femur) rather than between two rigid bodies (i.e., femur and tibia).

Referring now to FIG. 13, another embodiment of a knee orthosis 20 is shown. In this embodiment, the femoral cuff 22 and the tibial cuff 24 are each fastened to the wearer's leg by a pair of strap assemblies 22′, 24′. The depicted arrangement can minimize the risk of vertical migration, i.e., where the orthosis 20 slides up or down the wearer's leg, for instance due to the combination of gravity and vibrations caused by movements of the wearer. In this embodiment, the strap assemblies 24′ for the tibial cuff 24 are respectively positioned at upper and lower portions of the wearer's calf so that the bulging nature of the calf prevents vertical migration of the orthosis 20. Various angles for the strap assemblies 24′ are contemplated. In an exemplary embodiment, the upper strap assembly 24′ is angled at approximately 10 degrees relative to the horizontal in a downward direction from the rear to the front of the wearer's leg, and the lower strap assembly 24′ is angled at approximately 20 degrees relative to the horizontal in an upward direction from the rear to the front of the wearer's leg, Other means for minimizing the risk of vertical migration are contemplated.

The implementations described in this document provide non-limiting examples of possible implementations of the present technology. Upon review of the present disclosure, a person of ordinary skill in the art will recognize that changes may be made to the implementations described herein without departing from the scope of the present technology.

1. A knee orthosis comprising:
a femoral cuff configured for engaging an upper leg portion of a wearer;
a tibial cuff configured for engaging a lower leg portion of the wearer;
a hinge pivotally connecting the femoral cuff to the tibial cuff, the hinge including a tibial hinge member connected to a tibial cuff member of the tibial cuff; and
a varus-valgus adjustment system including:
a single pivot defining a pivot axis extending in a sagittal plane, the single pivot pivotally connecting the tibial hinge member to the tibial cuff member about the pivot axis;
an adjustment screw rotatably connected to the tibial hinge member; and
a block threadably engaged to the adjustment screw, the block comprising a protrusion projecting from the block and engaging a guide recess of the tibial cuff member or an intermediate linkage attached to the tibial cuff member; and
wherein a varus-valgus angle of the tibial cuff relative to the femoral cuff is selected by rotating the adjustment screw, thereby causing the protrusion to slide in the guide recess and pivoting the tibial cuff member relative to the tibial hinge member about the single pivot between a first position and a second position different from the first position.

2. The knee orthosis of claim 1, wherein the guide recess extends in a frontal plane perpendicular to the sagittal plane at an angle comprised between 20 and 40 degrees relative to a lateral direction in the frontal plane.

3. The knee orthosis of claim 1, wherein the adjustment screw threadably engages the block via a plurality of threads, each of the threads of the adjustment screw engaging the block being surrounded by complementarily shaped threads defined in the block.

4. The knee orthosis of claim 1, wherein the protrusion is removably connected to the block.

5. The knee orthosis of claim 1, wherein, when the knee orthosis is worn by the wearer, the single pivot is adapted to be located at about a tibial plateau of the wearer.

6. The knee orthosis of claim 1, wherein the single pivot is located vertically below the hinge.

7. The knee orthosis of claim 1, wherein, in the first position, the tibial cuff member is pivoted to the tibial hinge member by an angle of at most-45 degrees relative to the sagittal plane, and in the second position, the tibial cuff member is pivoted to the tibial hinge member by an angle at most of +45 degrees relative to the sagittal plane.

8. The knee orthosis of claim 7, wherein the tibial cuff member is pivoted to the tibial hinge member and repositionable relative thereto at any angular position between-25 degrees and +25 degrees relative to the sagittal plane.

9. The knee orthosis of claim 1, wherein:
the hinge includes a femoral portion and a tibial portion, the tibial portion including the tibial hinge member, the femoral portion and the tibial portion including corresponding shells engaged with one another and configured for pivot relative to one another, the hinge defining an axis intersecting a center of rotation of a knee of the wearer when the knee orthosis is worn by the wearer.

10. The knee orthosis of claim 9, wherein the center of rotation is an instantaneous center of rotation at a given flexion configuration of the hinge, the instantaneous center of rotation moving along a trajectory during flexion of the hinge, and the axis intersecting the instantaneous center of rotation notwithstanding the flexion configuration of the hinge.

11. The knee orthosis of claim 10, wherein the trajectory is contained in a plane parallel to the sagittal plane.

12. The knee orthosis of claim 1, wherein the adjustment screw defines a longitudinal screw axis about which the adjustment screw rotates, the longitudinal screw axis extending in a frontal plane orthogonal to the sagittal plane and generally parallel to the sagittal plane.

13. A knee orthosis comprising:
a femoral cuff configured for engaging an upper leg portion of a wearer;
a tibial cuff configured for engaging a lower leg portion of the wearer;
a hinge pivotally connecting the femoral cuff to the tibial cuff, the hinge including a femoral portion and a tibial portion, the femoral portion including a femoral hinge member connected to a femoral cuff member of the femoral cuff, the tibial portion including a tibial hinge member connected to a tibial cuff member of the tibial cuff; and
a varus-valgus adjustment system including:
a single pivot defining a pivot axis extending in a sagittal plane, the single pivot pivotally connecting either the tibial hinge member to the tibial cuff member or the femoral hinge member to the femoral cuff member for relative pivoting therebetween about the pivot axis;
an adjustment screw rotatably interconnecting either the tibial hinge member to the tibial cuff member or the femoral hinge member to the femoral cuff member, the adjustment screw defining a longitudinal screw axis extending in a frontal plane orthogonal to the sagittal plane and generally parallel to the sagittal plane; and
wherein a varus-valgus angle between the tibial cuff and the femoral cuff is selected by rotating the adjustment screw, thereby pivoting either the tibial cuff member relative to the tibial hinge member or the femoral cuff member relative to the femoral hinge member about the single pivot between a first position and a second position different from the first position.

14. A knee orthosis for a knee comprising:
a femoral cuff configured for engaging an upper leg portion of a wearer;
a tibial cuff configured for engaging a lower leg portion of the wearer;
a hinge pivotally connecting the femoral cuff to the tibial cuff and configured to allow flexion-extension of the knee, the hinge including a first hinge member pivotable relative to a second hinge member, the first hinge member and the second hinge member each connected to a respective cuff member of the tibial cuff and the femoral cuff; and
a varus-valgus adjustment system including:
a single pivot defining a pivot axis extending in a sagittal plane, the single pivot pivotally connecting one of the first and second hinge members with the respective cuff member for relative pivoting therebetween about the pivot axis;
an actuating element;
a translating element engaged with the actuating element, the translation element and the actuating element each being operatively connected to a respective one of: said one of the first and second hinge members; and the respective cuff member;
wherein the translating element engages a guiding feature forming part of or being attached to the other of: said one of the first and second hinge members; and the respective cuff member; the guiding feature defining a displacement path of the translating element; and
wherein a varus-valgus angle of the tibial cuff relative to the femoral cuff is adjusted by effecting displacement of the translating element, by way of the actuating element, guided by the displacement path, thereby pivoting the respective cuff member relative to the one of the first and second hinge members about the single pivot between a first position and a second position different from the first position.

15. The knee orthosis of claim 14, wherein the actuating element includes an adjustment screw rotatably connected to the tibial hinge member.

16. The knee orthosis of claim 15, wherein the translating element includes a block threadably engaged to the adjustment screw, the block comprising a protrusion projecting from the block and engaging the guiding feature of the tibial cuff member or an intermediate linkage attached to the tibial cuff member, wherein the varus-valgus angle of the tibial cuff relative to the femoral cuff is selected by rotating the adjustment screw, thereby causing the protrusion to slide in the guiding feature and pivoting the tibial cuff member relative to the tibial hinge member about the single pivot between the first position and the second position different from the first position.

17. The knee orthosis of claim 14, wherein the guiding feature extends in a frontal plane perpendicular to the sagittal plane at an angle comprised between 20 and 40 degrees relative to a lateral direction in the frontal plane.

18. The knee orthosis of claim 16, wherein the adjustment screw threadably engages the block via a plurality of threads, each of the threads of the adjustment screw engaging the block being surrounded by complementarily shaped threads defined in the block.

19. The knee orthosis of claim 16, wherein the protrusion is removably connected to the block.

20. The knee orthosis of claim 14, wherein, when the knee orthosis is worn by the wearer, the single pivot is located at about a tibial plateau of the wearer.

21. The knee orthosis of claim 14, wherein the single pivot is located vertically below the hinge.

22. The knee orthosis of claim 14, wherein, in the first position, the tibial cuff member is pivoted to the tibial hinge member by an angle of at most −45 degrees relative to the sagittal plane, and in the second position, the tibial cuff member is pivoted to the tibial hinge member by an angle at most of +45 degrees relative to the sagittal plane.

23. The knee orthosis of claim 22, wherein the tibial cuff member is pivoted to the tibial hinge member and repositionable relative thereto at any angular position between −25 degrees and +25 degrees relative to the sagittal plane.

24. The knee orthosis of claim 14, wherein a locking element is operably engaged with the translating element and/or the actuating element, the locking element being operable to selectively retain the translating element in a fixed position relative to the displacement path so as to maintain a selected varus-valgus angle.
